(19) United States
(12) Patent Application Publication
(10) Pub. No.: US 2011/0277752 A1
(43) Pub. Date: Nov. 17, 2011

(54) RECEPTACLE FOR AN AEROSOLIZABLE PHARMACEUTICAL FORMULATION

(75) Inventors: Scot Cheu, San Jose, CA (US); Leo Chan, Fremont, CA (US)

(73) Assignee: NOVARTIS AG, Basel (CH)

(21) Appl. No.: 12/745,365

(22) PCT Filed: Dec. 4, 2008
(86) PCT No.: PCT/US2008/013438

§ 371 Date: May 24, 2011

Related U.S. Application Data
(60) Provisional application No. 61/005,487, filed on Dec. 5, 2007.

(51) Int. Cl. A61M 11/00

(52) U.S. Cl. 128/200.14

(57) ABSTRACT

An article for storing a pharmaceutical formulation. In one or more embodiments, the present invention comprises a receptacle (125) for an aerosolizable formulation, the receptacle having a wall thickness of between about 100-235 microns, wherein the receptacle is puncturable to allow escape and dispersion of the formulation therein. Also provided are methods of aerosolizing formulations for inhalation, and systems for aerosolizing such formulations.

### BACKGROUND OF THE INVENTION

The need for effective therapeutic treatment of patients has resulted in the development of a variety of pharmaceutical formulation delivery techniques. One traditional technique involves the oral delivery of a pharmaceutical formulation in the form of a pill, capsule, elixir, or the like. However, oral delivery can in some cases be undesirable. For example, many pharmaceutical formulations may be degraded in the digestive tract before they can be effectively absorbed by the body. Inhalable drug delivery, where an aerosolized pharmaceutical formulation is orally or nasally inhaled by a patient to deliver the formulation to the patient's respiratory tract, has proven to be a particularly effective and/or desirable alternative. For example, in one inhalation technique, an aerosolized pharmaceutical formulation provides local therapeutic relief to a portion of the respiratory tract, such as the lungs, to treat diseases such as asthma, emphysema, and cystic fibrosis. In another inhalation technique, a pharmaceutical formulation is delivered deep within a patient's lungs where it may be absorbed into the blood stream. Many types of inhalation devices exist including devices that aerosolize a dry powder pharmaceutical formulation.

One type of inhalation device aerosolizes a formulation, such as an active agent or pharmaceutical, that is stored in a capsule. For example, a dose or a portion of a dose of a powder pharmaceutical formulation may be stored in a capsule, and the capsule may be inserted into an aerosolization device which is capable of aerosolizing the pharmaceutical formulation. After being inserted into the aerosolization device, the capsule is opened to expose the pharmaceutical formulation. The opening of the capsule may be performed, for example, by puncturing, cutting or tearing the capsule. When the capsule is properly opened and when aerosolization energy is supplied, the pharmaceutical formulation is aerosolized so that it may be inhaled by the user and a dose or portion of a dose of the aerosolized pharmaceutical formulation may be delivered to the user's respiratory tract.

However, improper use of the aerosolization device may result in the delivery of less than the desired amount of the pharmaceutical formulation. For example, if a capsule is not properly or completely opened before the aerosolization process, the amount of pharmaceutical formulation aerosolized may be reduced or the flow of the aerosolized pharmaceutical formulation may not be sufficient to deliver a desirable amount, such as a therapeutic amount, to the user. The effects of improper opening may be magnified when a user is unable or unwilling to visually inspect the opening of the capsule. The user may then unknowingly inhale less than a desired amount of the pharmaceutical formulation. In addition, sharpened elements for creating the opening in the capsule may produce inconsistent openings into the capsule which can result in inconsistent delivery of aerosolized medicament.

Pharmaceutical grade capsules of the art often have a non-uniform wall thickness, often thicker at the end for reasons of mechanical durability. Such capsules often have variations in the wall thickness at the ends, and may vary capsule to capsule (as in large lots) or may vary from one end of a capsule to another, or both.

Therefore, it is desirable to be able to provide a receptacle for an aerosolizable pharmaceutical formulation that is readily and consistently openable, yielding a reliable and repeatable dose. It is further desirable to be able to provide such opening without the need for specifically designed cutting or puncturing elements. It is further desirable to provide such opening with a variety of capsule compositions, such as polymeric compositions, and over a range of receptacle storage conditions, such as temperature and humidity.

### SUMMARY OF THE INVENTION

One or more of the embodiments of the present invention satisfies one or more of these needs.

Thus, one or more embodiments of the present invention include puncturable receptacles adapted to contain aerosolizable formulations, the receptacles comprising one or more regions of a uniform wall thickness, and/or a uniform range of wall thicknesses, wherein at least one of said regions a uniform wall thickness, and/or a uniform range of wall thicknesses comprises a situs of puncturing. Also provided are of aerosolizable formulations for inhalation, and systems for aerosolizing formulations for inhalation. Other features and advantages of embodiments of the present invention will be set forth in the description of invention that follows, and in part will be apparent from the description or may be learned by practice of the invention.

In another aspect of the invention, an aerosolization system comprises an aerosolization device comprising a chamber adapted to receive a receptacle. The aerosolization system also comprises a receptacle containing a pharmaceutical formulation, the receptacle comprising a wall portion that opens reliably when a puncturing or piercing means applies a predetermined puncturing force thereto.

In another aspect of the invention, a method of aerosolizing a pharmaceutical formulation comprises providing an aerosolization device comprising a chamber; providing a receptacle containing a pharmaceutical formulation, the receptacle comprising a wall having one or more regions comprising a uniform thickness of between about 100 and 235 microns; applying a puncturing force to the one or more regions comprising a uniform thickness of the receptacle to create one or more openings therein; and aerosolizing the pharmaceutical formulation in the chamber.

In another aspect of the invention, an aerosolization apparatus comprises a capsule comprising a wall having a substantially uniform thickness of between about 100 and 235 microns, a housing defining a chamber having one or more air inlets, the chamber being sized to receive a capsule which contains an aerosolizable pharmaceutical formulation; a puncturing mechanism within the housing and comprising a puncture member, wherein the puncture member comprises a forward end shaped to form a cutting edge that is effective in cutting the substantially uniformly thick wall of the capsule to create an opening into the capsule; and an end section associated with the housing, the end section sized and shaped to be received in a user's mouth or nose so that the user may inhale through the end section to inhale aerosolized pharmaceutical formulation that has exited the capsule through the opening created in the capsule.

In another aspect of the invention, a method of aerosolizing a pharmaceutical formulation comprises providing a capsule comprising a wall having a substantially uniform thickness of between about 100 and 235 microns, the capsule containing an aerosolizable pharmaceutical formulation; advancing a puncture member through the substantially uniformly thick wall of the capsule to create an opening in the capsule, wherein the puncture member comprises a forward end shaped to form a cutting edge, wherein an opening into the capsule is created without a piece of the wall of the capsule becoming detached from the capsule; aerosolizing the pharmaceutical formulation by flowing air through the chamber; and administering the aerosolized pharmaceutical formulation to the respiratory tract of a user during the user's inhalation.

In one or more aspects of the invention a capsule comprises a wall having a substantially uniform thickness of between about 100 and 235 microns, the capsule containing an aerosolizable pharmaceutical formulation is provided for use with an inhaler device having a capsule opening member that has a sharpened leading end and an unsharpened trailing end to improve the effectiveness of a capsule puncture.

In another aspect of the invention, an aerosolization system comprises a capsule comprising a wall having a substantially uniform thickness of between about 100 and 235 microns, and a housing defining a chamber having one or more air inlets, the chamber being sized to receive the capsule, the capsule adapted to contain an aerosolizable pharmaceutical formulation; a puncturing mechanism within the housing and comprising a puncture member, wherein the puncture member comprises a forward end shaped to form a cutting edge that is effective in cutting the wall of the capsule to create an opening into the capsule, and wherein the puncture member comprises a trailing end shaped so that it has a non-cutting surface that does not cut the wall of the capsule when the trailing end is inserted into the opening created by the forward end; and an end section associated with the housing, the end section sized and shaped to be received in a user's mouth or nose so that the user may inhale through the end section to inhale aerosolized pharmaceutical formulation that has exited the capsule through the opening created in the capsule.

One or more embodiments the present invention comprises capsules adapted to contain aerosolizable formulations, the capsules having dome-shaped upper and lower portions, wherein said upper portion or lower portion, or both comprise regions of a uniform wall thickness, and/or a uniform range of wall thicknesses, and wherein comprises said upper portion or lower portion, or both comprise a situs of puncture.

In another aspect of the invention, a method of aerosolizing a pharmaceutical formulation comprises providing a capsule which comprises a wall having a substantially uniform thickness of between about 100 and 235 microns, the capsule containing an aerosolizable pharmaceutical formulation; advancing a puncture member through the substantially uniform wall of between about 100 and 235 microns of the capsule to create an opening in the capsule, wherein the puncture member comprises a forward end shaped to form a cutting edge and wherein the puncture member comprises a trailing end shaped so that it has a non-cutting surface that does not cut the wall of the capsule when the trailing end is inserted into the opening created by the forward end, wherein an opening into the capsule is created without a piece of the wall of the capsule becoming detached from the capsule; aerosolizing the pharmaceutical formulation by flowing air through the chamber; and administering the aerosolized pharmaceutical formulation to the respiratory tract of a user during the user's inhalation.

In one or more aspects of the invention, a receptacle is provided which is reliably and openable, and a plurality of such receptacles which are reliably and repeatably openable, without using a specially designed cutting or puncturing element, such as a cutting tip.

In one or more aspects, a capsule having one or more regions of a uniform wall thickness, and/or a uniform range of wall thicknesses, is provided along with a passive dry powder inhaler, wherein the inhaler comprises one or more piercing elements designed and configured to pierce the capsule about at least one of the capsule regions of uniform wall thickness and/or a uniform range of wall thicknesses.

In one or more aspects, a capsule having one or more regions of a uniform wall thickness, and/or a uniform range of wall thicknesses, is provided along with an active dry powder inhaler, wherein the inhaler comprises one or more piercing elements designed and configured to pierce the capsule about at least one of the capsule regions of uniform wall thickness and/or a uniform range of wall thicknesses.

In one or more aspects, a kit is provided, comprising at least one capsule having one or more regions of a uniform wall thickness, and/or a uniform range of wall thicknesses, and a dry powder inhaler, wherein the inhaler comprises one or more piercing elements designed and configured to pierce the capsule about at least one of the capsule regions of uniform wall thickness and/or a uniform range of wall thicknesses.

In one or more aspects, a kit is provided, comprising at least one capsule having one or more regions of a uniform wall thickness, and/or a uniform range of wall thicknesses, and a passive dry powder inhaler, which comprises one or more piercing elements designed and configured to pierce the capsule about at least one of the capsule regions of uniform wall thickness and/or a uniform range of wall thicknesses

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1A is a schematic sectional side view of an aerosolization apparatus and receptacle in an initial position;

FIG. 1B is a schematic sectional side view of the aerosolization apparatus and receptacle shown in FIG. 1A at the beginning a receptacle opening process;

FIG. 1C is a schematic sectional side view of the aerosolization apparatus and receptacle shown in FIG. 1A during a receptacle opening process;

FIG. 1D is a schematic sectional side view of the aerosolization apparatus and receptacle shown in FIG. 1A during the beginning of an aerosolization process;

FIG. 1E is a schematic sectional side view of the aerosolization apparatus and receptacle shown in FIG. 1A during the aerosolization process;

FIGS. 2A and 2B are schematic perspective views of a version of a receptacle according to the invention in an unopened and a partially opened condition, respectively;

FIGS. 2C and 2D are schematic perspective views of a version of a receptacle according to the invention in a partially opened and an opened condition, respectively;

FIGS. 3A through 3E are schematic sectional side views of a receptacle opening and aerosolization process using a receptacle according to the invention in another version of an aerosolization apparatus;

FIGS. 4A-4F are schematic side sectional views of puncturing members, or tips, in accordance with one or more embodiments of the present invention;

FIG. 5 is a close-up side view of a puncturing member in accordance with one or more embodiments of the present invention;

FIG. 6 is a close-up perspective view of a puncturing member in accordance with one or more embodiments of the present invention;

FIG. 7 is a close-up perspective view of a puncturing member in accordance with one or more embodiments of the present invention;

FIG. 8 is a close-up side view of a puncturing member, showing one version of a puncturing tip in accordance with one or more embodiments of the present invention;

FIG. 9 is a close-up perspective view of puncturing members in accordance with one or more embodiments of the present invention;

FIG. 10 is a schematic sectional side view of an embodiment of an aerosolization apparatus and receptacle of the present invention; and

FIG. 11 is a side view of an embodiment of an aerosolization apparatus of the present invention.

### DESCRIPTION OF THE INVENTION

It is to be understood that unless otherwise indicated the present invention is not limited to specific apparatus, structure, formulation components, drug delivery systems, manufacturing techniques, administration steps, or the like, as such may vary. In this regard, unless otherwise stated, a reference to a compound or component includes the compound or component by itself, as well as the compound in combination with other compounds or components, such as mixtures of compounds.

Before further discussion, a definition of the following terms will aid in the understanding of embodiments of the present invention.

As used herein, the singular forms “a,” “an,” and “the” include the plural reference unless the context clearly dictates otherwise. Thus, for example, reference to “a phospholipid” includes a single phospholipid as well as two or more phospholipids in combination or admixture unless the context clearly dictates otherwise.

Reference herein to “one embodiment”, “one version” or “one aspect” shall include one or more such embodiments, versions or aspects, unless otherwise clear from the context.

When referring to an active agent, the term encompasses not only the specified molecular entity, but also its pharmaceutically acceptable, pharmacologically active analogs, including, but not limited to, salts, esters, amides, hydrazides, N-alkyl derivatives, N-acyl derivatives, prodrugs, conjugates, active metabolites, and other such derivatives, analogs, and related compounds.

Unless otherwise noted, numerical wall thicknesses are mathematical means.

As used herein “active dry powder inhaler” refers to an inhalation device that does not rely solely on a patient's inspiratory effort to disperse and aerosolize a pharmaceutical composition contained within the device in a reservoir or in a unit dose form. Active dry powder inhalers include inhaler devices that comprise a means for providing energy to disperse and aerosolize the drug composition, such as pressurized gas, and/or vibrating or rotating elements.

As used herein, “passive dry powder inhaler” refers to an inhalation device that relies upon a patient's inspiratory effort to disperse and aerosolize a pharmaceutical composition contained within the device in a reservoir or in a unit dose form and does not include inhaler devices which comprise a means for providing energy, such as pressurized gas and/or vibrating or rotating elements, to disperse and aerosolize the drug composition. Passive inhalers thus use only the patient's inspiratory effort to provide aerosolization energy.

This application incorporates by reference the entire disclosures of US Patent Application Publication Numbers: 2005-0056280; 2005-0022813; 2003-0106827; 2005-0000518; and 2005-0150492, and U.S. application Ser. No. 10/821,652, all of which are commonly owned with the invention herein. Each patent application, patent application publication or patent, referred to herein is fully incorporated by reference hereby.

The present invention relates to an article for storing a pharmaceutical formulation. Although the article and process is illustrated in the context of storing an aerosolizable powder pharmaceutical or active agent formulation in a receptacle, the present invention can be used with or in other processes, systems, articles and components and should not be limited to the examples provided herein.

In one or more embodiments, the present invention comprises a receptacle for an aerosolizable pharmaceutical formulation, wherein the formulation is released by puncturing the receptacle, the receptacle having a substantially uniform wall thickness of at least about 100 microns wherein the region or regions of substantially uniform wall thickness are dimensioned and configured to align with a receptacle puncturing means.

In one or more embodiments, the present invention comprises a receptacle for an aerosolizable formulation, the receptacle having a wall thickness of between about 100-235 microns, wherein the receptacle is puncturable to allow escape and dispersion of the formulation therein.

In one or more embodiments, the present invention comprises a capsule for an aerosolizable pharmaceutical or active agent formulation, wherein the formulation is released by puncturing the capsule, the capsule having a wall thickness of between about 110-180 microns.

In one or more embodiments, the present invention comprises a receptacle for an aerosolizable pharmaceutical formulation, wherein the formulation is released by puncturing the receptacle, the receptacle having a substantially uniform wall thickness of between about 120-160 microns.

In one or more embodiments, the present invention comprises a plurality of capsules for an aerosolizable pharmaceutical or active agent formulation, wherein the formulation is released by puncturing a capsule or capsules, each of the plurality of capsules having a substantially uniform wall thickness of between about 120-160 microns.

In one or more embodiments, the present invention comprises a receptacle for an aerosolizable pharmaceutical formulation, wherein the formulation is released by puncturing the receptacle, the receptacle having a substantially uniform wall thickness which, at the site of puncturing, does not vary by more than about 10 microns.

In one or more embodiments, the present invention comprises a cellulosic capsule for an aerosolizable pharmaceutical formulation, wherein the formulation is released by puncturing the capsule, the capsule having a substantially uniform wall thickness of between about 110-180 microns.

In one or more embodiments, the present invention comprises an alkyl methyl cellulose capsule for an aerosolizable pharmaceutical formulation, wherein the formulation is released by puncturing the receptacle, the capsule having a wall thickness of between about 120-160 microns, and which, at the site of puncturing, does not vary by more than about 7 microns.

In one or more embodiments, the present invention comprises a receptacle for an aerosolizable formulation, the receptacle comprising one or more regions comprising a wall thickness of between about 100-235 microns, wherein at least one wall region is puncturable to allow escape and dispersion of the formulation therein.

In one or more embodiments, the present invention comprises a capsule for an aerosolizable pharmaceutical or active agent formulation, wherein the formulation is released by puncturing one or more wall regions of the capsule, the capsule comprising one or more regions comprising a wall thickness of between about 110-180 microns.

In one or more embodiments, the present invention comprises a receptacle for an aerosolizable pharmaceutical formulation, wherein the formulation is released by puncturing the receptacle, the receptacle comprising one or more regions comprising a wall thickness of between about 120-160 microns.

In one or more embodiments, the present invention comprises a plurality of capsules for an aerosolizable pharmaceutical or active agent formulation, wherein the formulation is released by puncturing a capsule or capsules, each of the plurality of capsules comprising one or more regions comprising a wall thickness of between about 120-160 microns.

In one or more embodiments, the present invention comprises a receptacle for an aerosolizable pharmaceutical formulation, wherein the formulation is released by puncturing the receptacle, the receptacle comprising a puncturable region comprising a substantially uniform wall thickness which, at the site of puncturing, does not vary by more than about 10 microns.

In one or more embodiments, the present invention comprises a cellulosic capsule for an aerosolizable pharmaceutical formulation, wherein the formulation is released by puncturing the capsule, the capsule comprising a puncturable region comprising a substantially uniform wall thickness of between about 110-180 microns.

In one or more embodiments, the present invention comprises a system for aerosolizing powder active agents, such as pharmaceuticals, the system comprising a housing defining a chamber having one or more air inlets, the chamber being sized to receive a capsule which contains an aerosolizable pharmaceutical formulation; a puncturing means within the housing and comprising a puncture member, wherein the puncture member comprises a forward end shaped to form a cutting edge that is effective in cutting the wall of the capsule to create an opening into the capsule; and an end section associated with the housing, the end section sized and shaped to be received in a user's mouth or nose so that the user may inhale through the end section to inhale aerosolized pharmaceutical formulation that has exited the capsule through the opening created in the capsule holding means, wherein the formulation is released by puncturing the receptacle, the receptacle having a wall thickness of between about 100-180 microns, and which, at the site of puncturing, does not vary by more than about 10 microns.

In one or more embodiments, the present invention comprises a method for aerosolizing a pharmaceutical formulation, the method comprising filling the formulation into a receptacle, such as a capsule, the receptacle having a uniform wall thickness, at a site of puncturing, of between about 100-180 microns, placing the receptacle into a chamber, advancing a puncturing means into the receptacle whereby a wall is punctured and whereby a contents thereof are released for inhalation.

In one or more embodiments, the present invention comprises a receptacle for an aerosolizable pharmaceutical formulation, wherein the formulation is released by puncturing the receptacle, the receptacle having a wall thickness of between about 100-240 microns, and which, at the site of puncturing, does not vary by more than 15 microns.

In one or more embodiments, the present invention comprises a plurality of cellulosic capsules for an aerosolizable pharmaceutical formulation, wherein the formulation is released by puncturing the capsule, each capsule having a uniform wall thickness of between about 110-180 microns, and wherein the wall thickness does not vary by more than about 10 microns among or between capsules.

In one or more embodiments, the present invention comprises a plurality of cellulosic capsules for an aerosolizable pharmaceutical formulation, wherein the formulation is released by puncturing the capsule, each capsule having a uniform wall thickness of between about 100-240 microns, and wherein a distribution of capsules is such that at least 99.7% of the capsules have a wall thickness between about 100 and about 240 microns.

In one or more embodiments, the present invention comprises a plurality of cellulosic capsules for an aerosolizable pharmaceutical formulation, wherein the formulation is released by puncturing the capsule, each capsule having a uniform wall thickness of between about 100-240 microns, and wherein a distribution of capsules is such that at least 95% of the capsules have a wall thickness between about 105 and about 225 microns.

In one or more embodiments, the present invention comprises a cellulosic capsule for an aerosolizable pharmaceutical formulation, wherein the formulation is released by puncturing the capsule with a puncturing means, the capsule having a uniform wall thickness of between about 120-160 microns, and wherein the puncturing means comprises any form of sharpened means, such as a pointed element, an edged element, or combination thereof.

In one or more embodiments, the receptacle comprises a cellulosic or polymeric material.

In one or more embodiments, the receptacle comprises an alkyl cellulose, or hydroxy alkyl cellulose, material.

In one or more embodiments, the receptacle comprises a dome or hemispherical portion.

In one or more embodiments, the receptacle comprises an oval-shape.

In one or more embodiments, the receptacle comprises a spherical-shape.

In one or more embodiments, the receptacle comprises an ellipsoidal-shape.

In one or more embodiments, a situs of puncturing of the receptacles is about a curved or hemispherical wall portion.

In one or more embodiments, a situs of puncturing of the receptacles is about a straight wall portion.

In one or more embodiments a region of the receptacle comprises the entire receptacle.

Further embodiments of the present invention comprise two or more of any of the foregoing features, aspects, versions or embodiments.

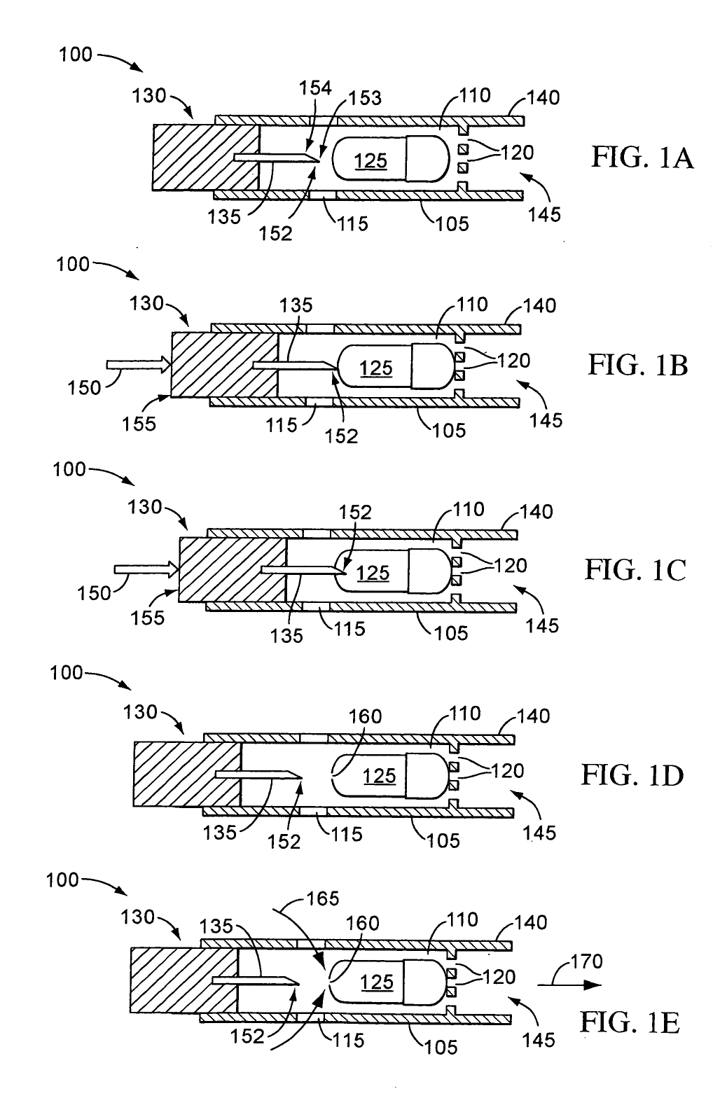

One embodiment of an aerosolization apparatus according to the present invention is shown schematically in FIGS. 1A-1E and is represented by the reference numeral 100, with a pharmaceutical formulation receptacle or capsule 125 The aerosolization apparatus 100 comprises a housing 105 defining a chamber 110 having one or more air inlets 115 and one or more air outlets 120. The chamber 110 is sized to receive a receptacle 125 which contains an aerosolizable pharmaceutical formulation. An opening mechanism 130 comprises an opening, or puncturing, member 135 that is moveable within the chamber 110. Near or adjacent the outlet 120 is an end section 140 that may be sized and shaped to be received in a user's mouth or nose so that the user may inhale through an opening 145 in the end section 140 that is in communication with the outlet 120. Alternatively, the end section 140 is in fluidic communication with any suitable patient interface to permit inhalation and delivery of the pharmaceutical formulation.

The aerosolization apparatus 100 utilizes air flowing through the chamber 110 to aerosolize the pharmaceutical formulation in the receptacle 125. For example, FIGS. 1A through 1E illustrate the operation of a version of an aerosolization apparatus 100 where air flowing through the inlet 115 is used to aerosolize the pharmaceutical formulation and the aerosolized pharmaceutical formulation flows through the outlet 120 so that it may be delivered to the user through the opening 145 in the end section 140. The aerosolization apparatus 100 is shown in its initial condition in FIG. 1A. The receptacle 125 is positioned within the chamber 110 and the pharmaceutical formulation is contained within the receptacle 125.

To use the aerosolization apparatus 100, the pharmaceutical formulation in the receptacle 125 is exposed to allow it to be aerosolized. In the version of FIGS. 1A though 1E, the opening mechanism 130 is advanced within the chamber 110 by applying a force 150 to the opening mechanism 130. For example, a user may press against a lower surface of the opening mechanism 130 to cause the opening mechanism 130 to slide within the housing 105 so that the opening, or puncturing, member 135 contacts the receptacle 125 in the chamber 110, as shown in FIG. 1B. By continuing to apply the force 150, the opening member 135 is advanced to abut the forward wall 122 of the receptacle 125, as shown in FIG. 1C. The opening member may comprise one or more tips 152 (which may be pointed, sharpened, angular, faceted or blunt) that contact the receptacle 125 in a manner that provides an opening into the receptacle 125. The opening mechanism 130 is then retracted to the position shown in FIG. 1D, leaving an opening 160 through the wall of the receptacle 125 to expose the pharmaceutical formulation in the receptacle 125.

Air or other gas then flows through an inlet or inlets 115, as shown by arrows 165 in FIG. 1E. The flow of air causes the pharmaceutical formulation to be aerosolized. When the user inhales (resulting in airflow represented by arrow 170 in FIG. 1E) through the end section 140 the aerosolized pharmaceutical formulation is delivered to the user's respiratory tract. In one version, the air flow 165 may be caused by the user's inhalation. In another version, compressed air or other gas may be ejected into the inlet 115 to cause the aerosolizing air flow 165.

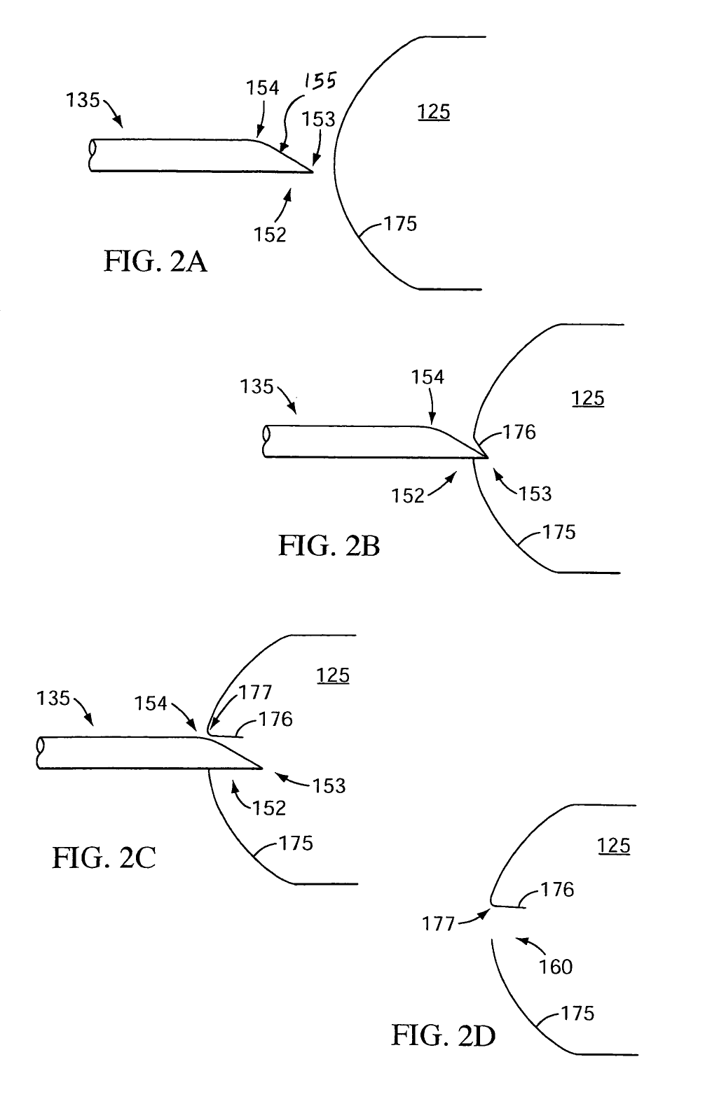

To increase the efficiency and effectiveness of the aerosolization apparatus 100, the puncture member 135 may comprise a tip 152 which is sharpened, having a forward end 153, a trailing end 154, and an intermediated planar portion 155 therebetween (shown in FIG. 2). The forward end 153 is shaped to form a cutting point or edge that is effective in cutting the wall of the capsule 125. Such shape comprises, in one or more embodiments, an elliptical or partially ellipsoidal shape, formed by an angled slice through a round cross-section of the member 135. In one or more embodiments, the trailing end 154 is shaped so that it has a non-cutting surface. For example, in one version, the trailing end 154 may be ground so that it has a smooth profile, as shown in FIG. 2A. FIGS. 2A through 2D illustrate the capsule puncturing process using one embodiment of a puncture member 135 of the present invention. As the puncture member 135 is advanced from the position shown in FIG. 2A to the position shown in FIG. 2B, the cutting tip on the forward end 153 cuts a wall 175 of the capsule. Continued advancement of the puncturing member 135, as shown in FIG. 2C, pushes a flap 176 of wall material inward into the capsule 125. Because of the non-cutting profile of the trailing end 154, the portion 177 of the flap 176 opposite the initial cut portion is bent and plastically deformed rather than being cut, leaving the opening 160 as shown in FIG. 2D when the puncture member 135 is retracted.

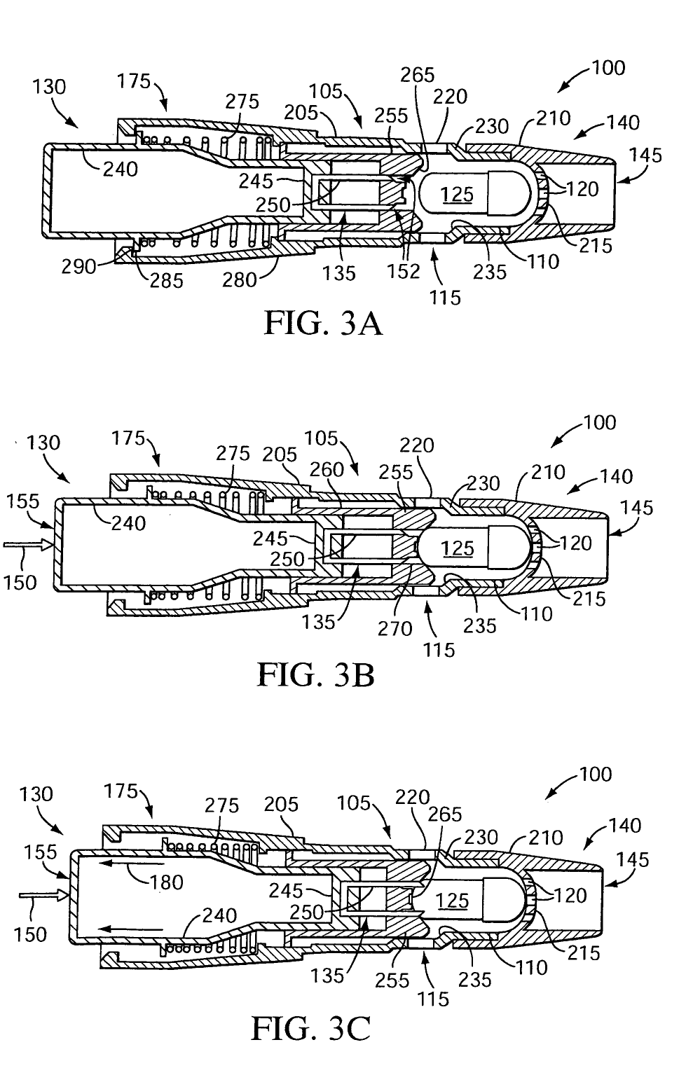

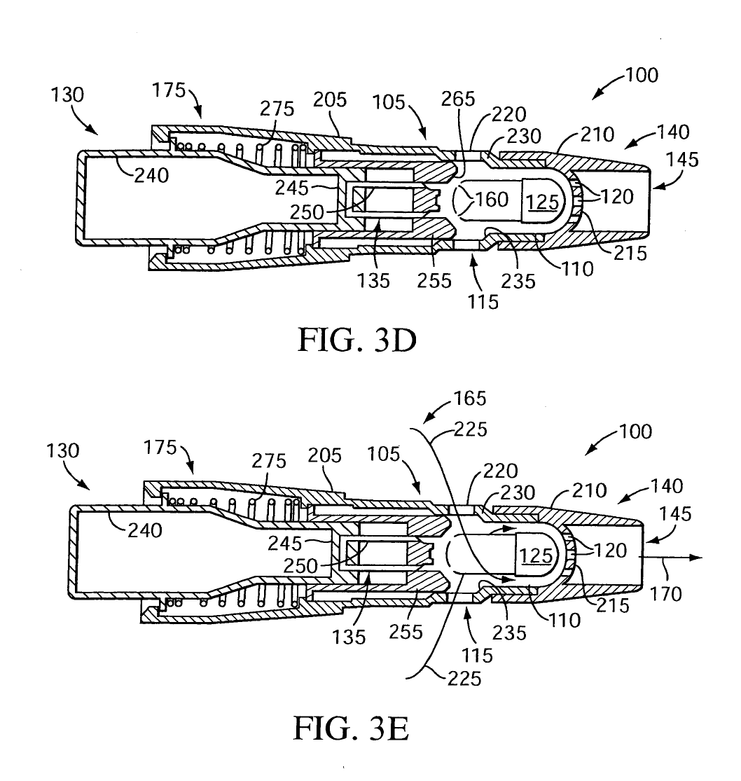

FIGS. 3A-3E show an example of an aerosolization apparatus with a chamber 110 as more fully described in U.S. Pat. No. 4,069,819 and in U.S. Pat. No. 4,995,385, both of which are incorporated herein by reference in their entireties. In such an arrangement, the chamber 110 comprises a longitudinal axis that lies generally in the inhalation direction, and the receptacle 125 is insertable lengthwise into the chamber 110 so that the receptacle's longitudinal axis may be parallel to the longitudinal axis of the chamber 110. In the version of FIGS. 3A through 3E, the chamber 110 is sized to receive a receptacle 125 containing a pharmaceutical formulation in a manner which allows the receptacle to move within the chamber 110. The plurality of openings 160 in the rear of the receptacle 125 in the version of FIGS. 3A through 3E are created by the opening mechanism 130 that is slidably disposed within a body 205.

The inlets 115 may comprise a plurality of tangentially oriented slots 220. When a user inhales (arrow 170 of FIG. 1E) through an endpiece 210, outside air is caused to flow through the tangential slots 220 as shown by arrows 225 in FIG. 3E. This airflow 225 creates a swirling airflow within the chamber 110. The swirling airflow causes the receptacle 125 to contact a partition 215 (incorporating one or more outlets 120) and then to move within the chamber 110 in a manner that causes the pharmaceutical formulation to exit the receptacle 125 and become entrained within the swirling airflow. In one or more versions, the partition 215 is dome-shaped, or hemispherical. In one or more versions, the receptacle 125 may rotate within the chamber 110 in a manner where the longitudinal axis of the receptacle, which may be a capsule, remains at an angle less than 80 degrees, and preferably less than 45 degrees from the longitudinal axis of the chamber. The movement of the receptacle 125 in the chamber 110 may be caused by the width of the chamber 110 being less than the length of the receptacle 125. In one specific version, the chamber 110 comprises a tapered section 230 that terminates at an edge 235. During the inspiratory flow of swirling air in the chamber 110, the forward end of the receptacle 125 may contact and rests upon a partition 215, and a sidewall of the receptacle 125 may contact the edge 235 and slides and/or may rotate along the edge 235. This motion of the receptacle 125, which may be a capsule, is particularly effective in forcing a large amount of the pharmaceutical formulation through the plurality of openings 160 in the rear of the receptacle 125.

The opening mechanism 130, shown in its rest position in FIG. 3A, comprises a plunger 240 attached at its forward end 245 to the opening member 135, which in the version shown is a puncturing member comprising a U-shaped staple 250 having a plurality of tips 152, such as the two tips shown in this version. The opening mechanism 130 further comprises a seating member (also referred to sometimes as an alignment guide) 255 which contacts the plunger 240 and/or the opening member 135 and is slidable relative to the plunger 240 and the opening member 135. To create the openings 160 in the receptacle 125, the user applies a force 150 to the plunger 240, as shown in FIG. 3B, such as by pressing against the end of the plunger 240 with the user's finger or thumb. The force 150 causes the plunger to slide within the body 205. A slight frictional contact between the plunger 240 and a rear section 260 of the seating member 255 causes the seating member 255 to also slide within the body 205 until a forward seating surface 265 of the seating member 255 contacts the receptacle 125, as shown in FIG. 3B. The forward seating surface 265, which may be shaped to generally match the adjoining shape (such as arcuate) of the receptacle 125, secures the receptacle 125 between the seating member 255 and the partition 215, which may also be shaped to generally match the shape of the receptacle 125. The continued application of force 150 causes the plunger 240 and the opening member 135 to slide relative to the seating member 255, as shown in FIG. 3C, to advance the opening member 135 through openings 270 in the forward seating surface 265 and to the receptacle 125 to create the openings 160 as discussed above. Upon the removal of the force 150, a spring 275 or other biasing member urges the opening mechanism 130 back to its rest position. For example, the spring 275 may contact a shoulder 280 in the body 205 and press a flange 285 on the plunger 240 toward a rim 290 in the body 205. The frictional engagement between the plunger 240 and the seating member 255 also returns the seating member 255 to its retracted position.

In one or more embodiments of the aerosolization system 100 of the present invention, the pharmaceutical formulation in the capsule 125 is exposed to ambient air to allow it to be aerosolized. In the version of FIGS. 3A though 3E, the puncture mechanism 130 is advanced within the chamber 110 by applying a force 150 to the puncture mechanism 130. Initially, the seating member 255 and the puncture member 135 advance as a unit to the position shown in FIG. 3B. In this position, the seating surface 265, which is dimensioned and configured to be generally congruent to a receptacle wall, such as a lower arcuate capsule end, contacts the capsule 125, and acts to center the capsule 125 within the chamber 110, as well as to align it such that a long axis of the capsule 125 is parallel to a centerline of the device. This serves to align the capsule 125 for proper puncturing, thus ensuring optimal aerosolization of the contents. As the force 150 is continued, the puncture member 135 is advanced into and through the wall of the capsule 125. The puncturing mechanism 130 is then retracted to the position shown in FIG. 3A, leaving an opening or openings 160 through the wall of the capsule 125 to expose the pharmaceutical formulation in the capsule 125.

Proper creation of the opening 160 in the capsule 125 allows for efficient and effective delivery of the aerosolized pharmaceutical formulation to the user. In contrast, improper creation of the opening 160 can lead to inefficient and less effective delivery of the medicament to a user. Therefore a properly designed sharpened tip 152 can help in the creation of consistent openings in the capsule. Also, it is important to have a tip 152, such as a sharpened tip, that does not result in the portion of the wall of the capsule 125 that is removed to create the opening 160 from becoming broken off from the capsule 125 and thereby becoming one or more loose fragments. These fragments may be inhaled by the user, potentially causing discomfort.

The puncture member 135 having a sharpened tip 152 with a non-cutting trailing end 154 provides many advantages. For example, a conventional puncture member may be formed from round wire than is sheared or ground along a plane at the trailing end or may be formed in a manner where the sharpened tip includes a non-straight edge at the trailing end, such as a curved edge formed by using a diamond shaped wire. Referring again to FIG. 2, these conventional puncture members will sometimes result in a flap 176 being cut at the portion 177 thereby causing the flap 176 to be released from the wall 175 (such as an arcuate end portion) of the receptacle 125 and potentially aerosolized. By providing a non-cutting trailing end 154, the number of loose flaps 176 is significantly reduced and more consistent punctures result.

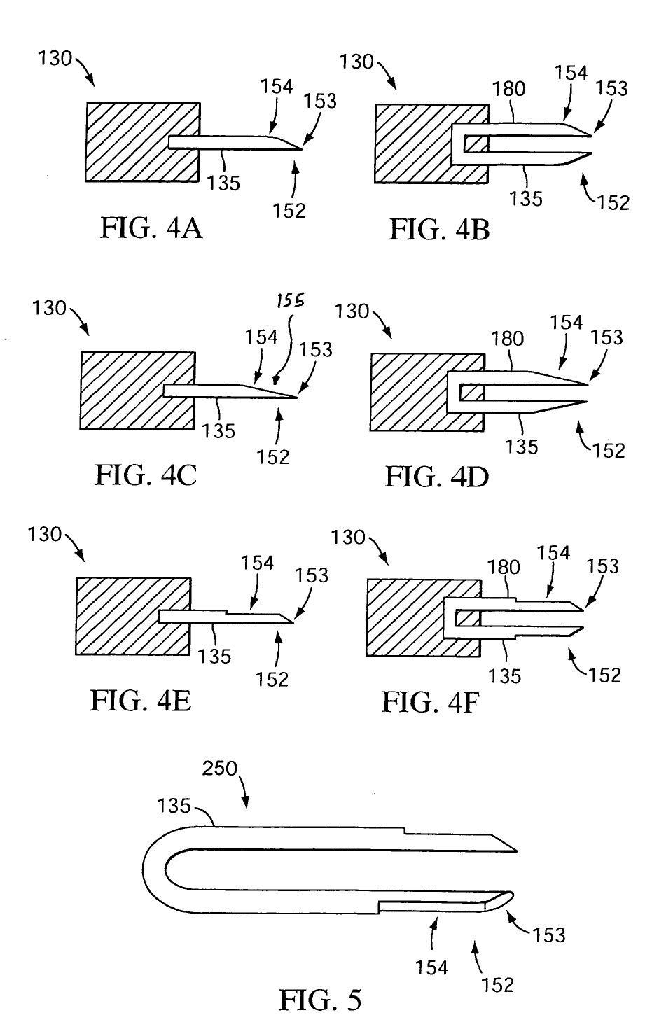

The non-cutting trailing end 154 of the sharpened tip 152 may be provided by grinding the trailing end 154, as discussed above, or by otherwise shaping the sharpened tip 152. Examples of sharpened tips 152 having non-cutting trailing ends are shown in FIGS. 4A, 4B, 4C, 4D, 4E, 4F, and 5-9. In the version of FIG. 4B, the two tips are provided on the opposite ends of the U-shaped puncture member 250. In the versions of FIGS. 4C and 4D, the sharpened tip 152 is provided by making a planar cut or grind in the puncture member 135. In this version, the cut is of sufficient length and/or angle that the trailing end 154 never contacts the capsule 125. Accordingly, only the forward end 153 and intermediate planar portion 155 contact the capsule, and the capsule is not subjected to the potentially deleterious effects of contact by the trailing edge 154. In some versions of the aerosolization apparatus, the advancement of the puncture member of FIGS. 4A and 4B is limited to prevent the exposure of the capsule to the trailing end 154.

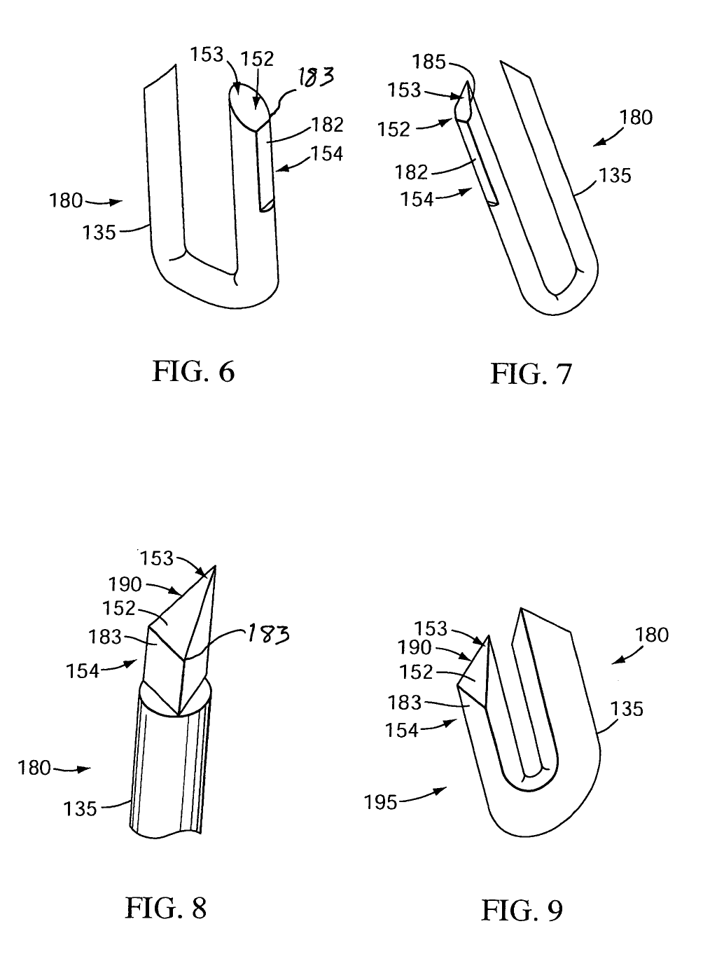

In one or more versions of FIG. 5, 6 or 7 a conventional round wire with a planar cut tip is further processed to cut away the trailing end 154 thereby removing the cutting portion of the trailing end, resulting in a planar surface 182, terminating in a straight edge 183. This provides a substantially D-shaped sharpened tip 152 as shown in FIGS. 5, 6 and 7. The planar surface 182 terminating in straight edge 183 is advantageous over a rounded or pointed edge of a conventional puncture member in reducing the number of loose flaps 176, in reducing the likelihood of the puncture member being captured within the capsule, and in reducing wear and tear on the aerosolization apparatus 100 in that the conventional edges often produce plastic shaving from contacted surfaces in the apparatus. The version of FIG. 7 is similar to the version of FIG. 6 but with one or more facets 185 being provided at the leading end 153 in order to facilitate advancement of the tip through the capsule wall 175. In the version of FIGS. 8 and 9, the sharpened tip 152 is formed into a substantially triangular shape 190. The planar surface 182 and straight edge 183 that results from the triangular shape 190 is advantageous is much the same manner as the planar surface 182 and straight edge 182 that results from the D-shape tip.

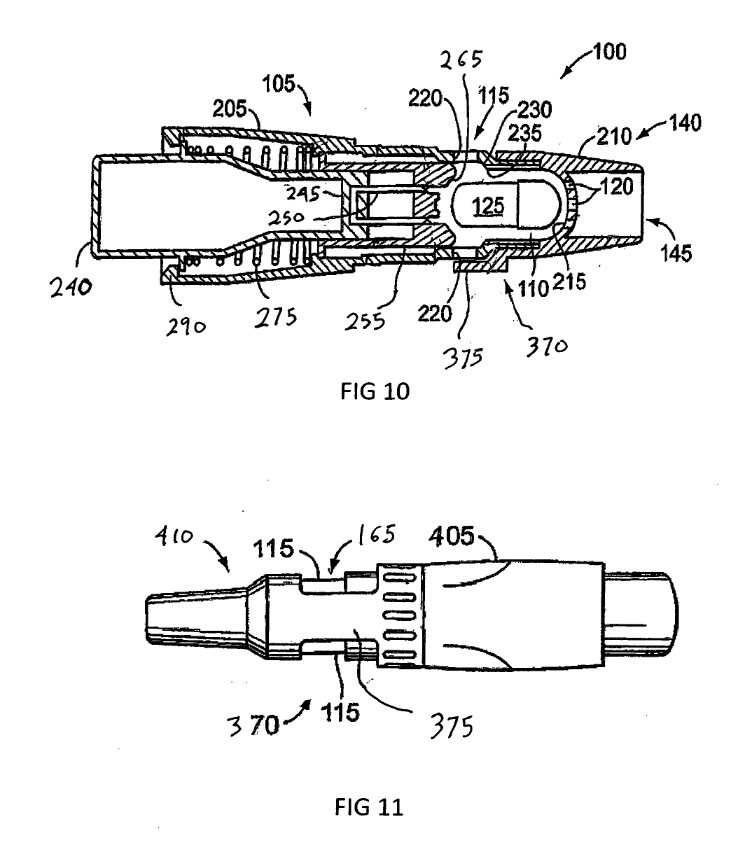

As shown in FIGS. 10 and 11, in one or more embodiments of an aerosolization system in accordance with the present invention, there is provided an air inlet shielding member 370 which comprises a covering portion 375 that at least partially covers one or more of the inlets 115. The shielding member 370 prevents blockage of the air flow by preventing at least one of the inlets 115 from being blocked by a user's fingers or hand during use. Accordingly, if a user inadvertently grasps the apparatus in the area of the inlets 115, the user will the shielding member 370 rather than one or more of the inlets 115 and air will still flow through into the chamber 110. As more fully described in WO 2004/091705, shielding member 370 and covering portion 375 may be dimensioned and configured such that the air flow 165 can take a more tortuous path in the region of the shielding member 370, or the shielding member 370 and/or covering portion 375 may be dimensioned and configured such that flow resistance is increased through the apparatus and coverage of all or a plurality of the inlets is desirable. In one or more versions, the shielding member 370 covers less than half of the inlets 115, affording ample air flow through the device, independent of user finger positioning. The term “cover” comprises an overlap in the radial or the outward direction, or both.

A version of an aerosolization apparatus 100 comprising a shielding member 370 is shown in FIG. 11. In this version, the housing 105 of the aerosolization apparatus 100 comprises a body 405 and a removable endpiece 410. The endpiece 410 may be removed from the body 405 to insert a receptacle 125 in the chamber 110 which is formed when the body 405 and the endpiece 410 are connected together.

It has been found that opening reliability and/or repeatability and/or shape integrity can be dependent upon one or more of wall thickness, wall thickness uniformity and wall thickness distribution for the receptacle 125. In one or more embodiments of the present invention, the receptacle has a uniform wall thickness of between about 100 and 240 microns. In one or more embodiments, a lower limit for the wall thickness is 100, or 105, or 110, or 115, or 120, or 125, or 130, or 135, or 140, or 145, or 150, or 155, or 160 microns. In one or more embodiments, an upper limit for the wall thickness is 240, or 235, or 230, or 225, or 220, or 215, or 210, or 205, or 200, or 195, or 190, or 185, or 180, or 175, or 170, or 165, or 160, or 155, or 150, or 145, or 140, or 135, or 130, or 125, or 120 microns. In one or more embodiments, a range of wall thicknesses is provided wherein any lower limit may be combined with any upper limit which is greater than the lower limit. In one or more embodiments, a range of wall thicknesses is provided wherein any upper limit may be combined with any lower limit which is lesser than the upper limit.

In one or more embodiments, any numerical value disclosed herein may be considered the midpoint of a size range wherein the range comprises a total of 5, 6, 7, 8, 9, 10, 11, 12, 13, 14, 15, 16, 17, 18, 19, 20, 25, 30, 35 or 40 microns. Midpoint values, unless otherwise specified, are mean values.

In one or more embodiments, the wall thickness is between about 130 and 155 microns.

In one or more embodiments, a plurality of capsules are provided wherein a distribution of wall thicknesses comprises at least about 99.7% are between about 100 and 235 microns; and/or at least about 95% are between about 105 and about 225 microns; and/or at least about 90% % are between about 110 and about 200 microns.

Each of the thickness ranges discussed herein may relate to the receptacle about its entire surface, or may relate only to that surface of the capsule which is desired to be pierced or punctured by the puncturing apparatus, for example wall 175 of FIG. 2.

In one or more embodiments, the receptacle comprises a capsule, and the surface to be punctured is the curved or hemispherical end surface, for example, as shown in FIGS. 1-3. In one or more embodiments, the end surface is defined by Equation I:

$$H ( x , y ) = \{ \begin{matrix}\sqrt{a - {x}^{2} - {y}^{2}} & for \sqrt{{x}^{2} + {y}^{2}} \leq a \\ 0 & for \sqrt{{x}^{2} + {y}^{2}} > a .\end{matrix} \qquad Equation I$$

In one or more embodiments, the curved end surface comprises the wall thickness ranges described herein.

In one or more embodiments, unexpected advantages occur with a uniform wall thickness range as defined herein. For example, the incidents of capsule denting, and concomitant reduction or absence of powder emptying and/or dispersion from the capsule, are minimized or eliminated. Capsule puncturing is more reliable and efficient, and the need for a specifically-designed cutting edge is minimized or eliminated. Thus the receptacles of the present invention may be reliably used with a variety of cutting edge designs or shapes, such as points, tapers, edges, and combinations thereof. In one or more embodiments, receptacle, such as capsule, puncturing is reliably achieved even if the puncturing surface is not completely smooth and free of imperfections or irregularities. In one or more embodiments, receptacle, such as capsule, puncturing is reliably achieved even if the puncturing surface is not completely aligned with the surface to be punctured.

In one or more embodiments, the various embodiments of uniform wall size ranges, and distributions reduce the deleterious effects of humidity on reliable and repeatable capsule puncturing.

In other versions, the aerosolization apparatus 100 may be configured differently than as shown in FIGS. 1A through 1E and 3A through 3E. For example, the chamber 100 may be sized and shaped to receive the receptacle 125 so that the receptacle 125 is orthogonal to the inhalation direction, as described in U.S. Pat. No. 3,991,761. As also described in U.S. Pat. No. 3,991,761, the opening mechanism 130 may contact both ends of the receptacle 125. In another version, the chamber may receive the receptacle 125 in a manner where air flows through the receptacle 125 as described for example in U.S. Pat. No. 4,338,931 and in U.S. Pat. No. 5,619,985. In another version, the aerosolization of the pharmaceutical formulation may be accomplished by pressurized gas flowing through the inlets, as described for example in U.S. Pat. No. 5,458,135, U.S. Pat. No. 5,785,049, and U.S. Pat. No. 6,257,233, or propellant, as described in PCT Publication WO 00/72904 and U.S. Pat. No. 4,114,615. All of the above references are incorporated herein by reference in their entireties.

In one or more versions of the present invention, the receptacle 125 comprises a capsule type receptacle. The capsule may be of a suitable shape, size, and material to contain the pharmaceutical formulation and to provide the pharmaceutical formulation in a usable condition. For example, the capsule may comprise a wall 175 (shown in FIGS. 2A-2D) which comprises a material that does not adversely react with the pharmaceutical formulation. In addition, the wall may comprise a material that allows the capsule to be opened to allow the pharmaceutical formulation to be aerosolized. In one version, the wall comprises one or more of gelatin, a cellulosic material such as alkyl or aryl methylcellulose, hydroxy alkyl methylcellulose, hydroxypropyl methylcellulose (HPMC), polyethyleneglycol-compounded HPMC, hydroxypropylcellulose, agar, polyvinyl alcohol, polyvinyl acetate, co-polymers thereof and combinations thereof. Alternatively or additionally, the capsule wall may comprise a polymeric material, such as polyvinyl chloride (PVC). Alternatively or additionally, the capsule wall may comprise a metal, such as aluminum.

In one or more versions, the capsule may comprise telescopically joined sections, as described for example in U.S. Pat. No. 4,247,066 which is incorporated herein by reference in its entirety. The interior of the capsule may be filled with a suitable amount of the pharmaceutical formulation, and the size of the capsule may be selected to adequately contain a desired amount of the pharmaceutical formulation. The sizes generally range from size 5 to size 000 with the outer diameters ranging from about 4.91 mm to 9.97 mm, the heights ranging from about 11.10 mm to about 26.14 mm, and the volumes ranging from about 0.13 ml to about 1.37 ml, respectively. Exemplary capsule sizes and corresponding volumes are shown in Table 1 below:

TABLE 1

| Capsule Size | 000 | 00 | 0 | 1 | 2 | 3 | 4 | 5 |
|---|---|---|---|---|---|---|---|---|
| Volume (mL) | 1.37 | 0.95 | 0.68 | 0.50 | 0.37 | 0.30 | 0.21 | 0.13 |

Suitable capsules are available commercially from, for example, Qualicaps Inc. in Whitsett, N.C. and Nara, Japan, and Capsugel in Greenwood, S.C. After filling, a top portion may be placed over the bottom portion to form the a capsule shape and to contain the powder within the capsule, as described in U.S. Pat. No. 4,846,876, U.S. Pat. No. 6,357,490, and in the PCT application WO 00/07572 published on Feb. 17, 2000, all of which are incorporated herein by reference in their entireties.

In one or more embodiments, the invention provides a system and method for aerosolizing a pharmaceutical formulation and delivering the pharmaceutical formulation to the respiratory tract of the user, and in particular to the lungs of the user. The pharmaceutical formulation may comprise powdered medicaments, liquid solutions or suspensions, and the like, and may include an active agent. In one or more embodiments, the system and method for aerosolizing a pharmaceutical formulation and delivering the pharmaceutical formulation includes one or more embodiments of the receptacle, such as capsule, described herein.

The active agent described herein comprises an agent, drug, compound, composition of matter or mixture thereof which provides some pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. An active agent for incorporation in the pharmaceutical formulation described herein may be an inorganic or an organic compound, including, without limitation, drugs which act on: the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system, and the central nervous system. Suitable active agents may be selected from, for example, hypnotics and sedatives, psychic energizers, tranquilizers, respiratory drugs, anticonvulsants, muscle relaxants, antiparkinson agents (dopamine antagnonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents. The active agent, when administered by inhalation, may act locally or systemically.

The active agent may fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of active agents suitable for use in this invention include but are not limited to one or more of calcitonin, amphotericin B, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), tacrolimus, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), parathyroid hormone analogs, parathyroid hormone fragments, glucagon-like peptide thymosin alpha 1, IIb/IIIa inhibitor, alpha-1 antitrypsin, phosphodiesterase (PDE) compounds, VLA-4 inhibitors, bisphosphonates, respiratory syncytial virus antibody, cystic fibrosis transmembrane regulator (CFTR) gene, deoxyribonuclease (Dnase), bactericidal/permeability increasing protein (BPI), anti-CMV antibody, 13-cis retinoic acid, macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate, polymixins such as polymixin B, capreomycin, bacitracin, penems; penicillins including penicllinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the invention is intended to encompass synthetic, native, glycosylated, unglycosylated, pegylated forms, and biologically active fragments and analogs thereof.

Active agents for use in the invention further include nucleic acids, as bare nucleic acid molecules, vectors, associated viral particles, plasmid DNA or RNA, siRNA, or other nucleic acid constructions of a type suitable for transfection or transformation of cells, i.e., suitable for gene therapy including antisense. Further, an active agent may comprise live attenuated or killed viruses suitable for use as vaccines. Other useful drugs include those listed within the Physician's Desk Reference (most recent edition).

The amount of active agent in the pharmaceutical formulation will be that amount necessary to deliver a therapeutically effective amount of the active agent per unit dose to achieve the desired result. In practice, this will vary widely depending upon the particular agent, its activity, the severity of the condition to be treated, the patient population, dosing requirements, and the desired therapeutic effect. The composition will generally contain anywhere from about 1% by weight to about 99% by weight active agent, typically from about 2% to about 95% by weight active agent, and more typically from about 5% to 85% by weight active agent, and will also depend upon the relative amounts of additives contained in the composition. The compositions of the invention are particularly useful for active agents that are delivered in doses of from 0.001 mg/day to 100 mg/day, preferably in doses from 0.01 mg/day to 75 mg/day, and more preferably in doses from 0.10 mg/day to 50 mg/day. It is to be understood that more than one active agent may be incorporated into the formulations described herein and that the use of the term “agent” in no way excludes the use of two or more such agents.

The pharmaceutical formulation may comprise a pharmaceutically acceptable excipient or carrier which may be taken into the lungs with no significant adverse toxicological effects to the subject, and particularly to the lungs of the subject. In addition to the active agent, a pharmaceutical formulation may optionally include one or more pharmaceutical excipients which are suitable for pulmonary administration. These excipients, if present, are generally present in the composition in amounts ranging from about 0.01% to about 95% percent by weight, preferably from about 0.5 to about 80%, and more preferably from about 1 to about 60% by weight. Preferably, such excipients will, in part, serve to further improve the features of the active agent composition, for example by providing more efficient and reproducible delivery of the active agent, improving the handling characteristics of powders, such as flowability and consistency, and/or facilitating manufacturing and filling of unit dosage forms. In particular, excipient materials can often function to further improve the physical and chemical stability of the active agent, minimize the residual moisture content and hinder moisture uptake, and to enhance particle size, degree of aggregation, particle surface properties, such as rugosity, ease of inhalation, and the targeting of particles to the lung. One or more excipients may also be provided to serve as bulking agents when it is desired to reduce the concentration of active agent in the formulation.

Pharmaceutical excipients and additives useful in the present pharmaceutical formulation include but are not limited to amino acids, peptides, proteins, non-biological polymers, biological polymers, carbohydrates, such as sugars, derivatized sugars such as alditols, aldonic acids, esterified sugars, and sugar polymers, which may be present singly or in combination. Suitable excipients are those provided in WO 96/32096, which is incorporated herein by reference in its entirety. The excipient may have a glass transition temperature (T_{g}) above about 35° C., preferably above about 40° C., more preferably above 45° C., most preferably above about 55° C.

Exemplary protein excipients include albumins such as human serum albumin (HSA), recombinant human albumin (rHA), gelatin, casein, hemoglobin, and the like. Suitable amino acids (outside of the dileucyl-peptides of the invention), which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and polypeptides that function as dispersing agents. Amino acids falling into this category include hydrophobic amino acids such as leucine, valine, isoleucine, tryptophan, alanine, methionine, phenylalanine, tyrosine, histidine, and proline. Dispersibility-enhancing peptide excipients include dimers, trimers, tetramers, and pentamers comprising one or more hydrophobic amino acid components such as those described above.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol and the like.

The pharmaceutical formulation may also include a buffer or a pH adjusting agent, typically a salt prepared from an organic acid or base. Representative buffers include organic acid salts of citric acid, ascorbic acid, gluconic acid, carbonic acid, tartaric acid, succinic acid, acetic acid, or phthalic acid, Tris, tromethamine hydrochloride, or phosphate buffers.

The pharmaceutical formulation may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-β-cyclodextrin and sulfobutylether-β-cyclodextrin), polyethylene glycols, and pectin.

The pharmaceutical formulation may further include flavoring agents, taste-masking agents, inorganic salts (for example sodium chloride), antimicrobial agents (for example benzalkonium chloride), sweeteners, antioxidants, antistatic agents, surfactants (for example polysorbates such as “TWEEN 20” and “TWEEN 80”), sorbitan esters, lipids (for example phospholipids such as lecithin and other phosphatidylcholines, phosphatidylethanolamines), fatty acids and fatty esters, steroids (for example cholesterol), and chelating agents (for example EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), both of which are incorporated herein by reference in their entireties.

“Mass median diameter” or “MMD” is a measure of mean particle size, since the powders of the invention are generally polydisperse (i.e., consist of a range of particle sizes). MMD values as reported herein are determined by centrifugal sedimentation, although any number of commonly employed techniques can be used for measuring mean particle size. “Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, generally in air, as the particle. The aerodynamic diameter encompasses particle shape, density and physical size of a particle. As used herein, MMAD refers to the midpoint or median of the aerodynamic particle size distribution of an aerosolized powder determined by cascade impaction.

In one or more versions, a powdered formulation for use in the present invention comprises a dry powder having a particle size selected to permit penetration into the alveoli of the lungs. In one or more versions, a powder size is less than about 20 μm (microns) mass median diameter (MMD), such as less than about 10 μm, less than about 8 μm, less than about 5 μm, or less than about 3 μm. In one or more versions, a powder size is in the range of about 0.1 μm to 12 μm in diameter (MMD), or about 1 μm to 8 μm in diameter (MMD). In one or more versions, a delivered dose efficiency (DDE) of these powders may be greater than about 30%, or greater than about 40%, or greater than about 50% or greater than about 60%, or greater than about 70%, or greater than about 80%.

In one or more versions, an aerodynamic powder size is less than about 8 μm (microns) mass median aerodynamic diameter (MMAD), or less than about 5 μm, or less than about 3 μm, or less than about 1 μm. In one or more versions an aerosol particle size distribution is about 0.3-8 μm mass median aerodynamic diameter (MMAD), such as about 0.5-5 μm MMAD, or about 1-4 μm MMAD, or about 1.5-3 μm MMAD. These dry powders have a moisture content below about 10% by weight, usually below about 5% by weight, and preferably below about 3% by weight. Such powders are described in WO 95/24183, WO 96/32149, WO 99/16419, and WO 99/16422, all of which are all incorporated herein by reference in their entireties.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the cooperating components may be reversed or provided in additional or fewer number. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

What is claimed is:

1. An aerosolization system comprising:
an aerosolization device comprising a housing having a chamber adapted to receive a receptacle;
a puncturing mechanism within the housing and comprising a puncture member, wherein the puncture member comprises a forward end shaped to form a cutting edge that is effective in cutting the wall of the capsule to create an opening into the receptacle; and
a receptacle containing a pharmaceutical formulation for inhalation, the receptacle comprising a wall comprising a substantially uniform thickness of between about 100 microns and about 240 microns,
whereby an opening into the receptacle may be created by applying a puncturing force to the receptacle, the opening of sufficient quality such that substantially all of the contents of the receptacle are discharged upon application of aerosolization energy.

2. The aerosolization system of claim 1 wherein,
the receptacle comprises a capsule.

3. The aerosolization system of claim 2 wherein,
the capsule comprises a wall thickness of between about 110 and 180 microns.

4. The aerosolization system of claim 2 wherein,
the capsule comprises a wall thickness of between about 120 and 160 microns.

5. The aerosolization system of claim 4 wherein,
the capsule comprises a wall thickness of between about 120 and 160 microns, and a wall uniformity of about 10 microns.

6. The aerosolization system of claim 5 wherein,
the wall thickness is uniform about a periphery of the capsule.

7. The aerosolization system of claim 5 wherein,
the wall thickness is uniform about a situs of puncturing of the capsule.

8. The aerosolization system of claim 7 wherein,
the situs of puncturing of the capsule comprises a hemispherical portion thereof.

9. A system according to claim 1 wherein the puncturing mechanism comprises a puncture member movable relative to the receptacle or to the chamber.

10. A system according to claim 1 wherein,
the receptacle comprises a wall comprising one or more of gelatin, hydroxypropyl methylcellulose, polyethyleneglycol-compounded hydroxypropyl methylcellulose, hydroxypropylcellulose, and agar.

11. A system according to claim 10 wherein,
the receptacle contains a powder pharmaceutical formulation.

12. A system according to claim 11 wherein,
the powder pharmaceutical formulation comprises particles having a mass median diameter less than about 20 μm.

13. A system according to claim 12 wherein,
the powder pharmaceutical formulation comprises particles having a mass median aerodynamic diameter less than about 10 μm.

14. A system according to claim 13 wherein the aerosolization device is a passive inhaler.

15. A receptacle for an aerosolizable formulation comprising
a capsule comprising one or more of gelatin, hydroxypropyl methylcellulose, polyethyleneglycol-compounded hydroxypropyl methylcellulose, hydroxypropylcellulose, and agar, and wherein a capsule wall thickness of between about 110-180 microns about a situs of puncturing, wherein the capsule is puncturable to allow escape and dispersion, solely by a patient's inspiratory effort, of the formulation therein.

16. The capsule of claim 15 wherein the capsule comprises a wall uniformity of about 10 microns.

17. The capsule of claim 16 wherein the capsule wall thickness is uniform about a periphery of the capsule.

18. A plurality of cellulosic capsules for containing an aersolizable pharmaceutical formulation, wherein the formulation is released by puncturing the capsule, each capsule comprising one or more of gelatin, hydroxypropyl methylcellulose, polyethyleneglycol-compounded hydroxypropyl methylcellulose, hydroxypropylcellulose, and agar, and comprising a uniform wall thickness of between about 110-180 microns, and wherein the wall thickness does not vary by more than about 20 microns among or between capsules.

19. A kit comprising:
a plurality of capsules, each comprising a wall having a uniform thickness of between about 100 and 235 microns, each capsule containing an aerosolizable pharmaceutical formulation comprising particles having a mass median aerodynamic diameter less than about 10 μm;
an aerosolization apparatus comprising a housing defining a chamber having one or more air inlets, the chamber being sized to receive said capsule;
a puncturing mechanism within the housing and comprising a puncture member, wherein the puncture member comprises a forward end shaped to form a cutting edge that is effective in cutting the wall of the capsule to create an opening into the capsule; and
an end section associated with the housing, the end section sized and shaped to be received in a user's mouth or nose so that the user may inhale through the end section to inhale aerosolized pharmaceutical formulation that has exited the capsule through the opening created in the capsule.

20. A method of aerosolizing a pharmaceutical formulation comprising
providing an aerosolization device comprising a chamber;
providing a receptacle containing a pharmaceutical formulation, the receptacle comprising a wall having a uniform thickness of between about 100 and 235 microns;
applying a puncturing force to the receptacle to create one or more openings therein; and
aerosolizing the pharmaceutical formulation in the chamber.
